(19) United States
(12) Patent Application Publication
(10) Pub. No.: US 2024/0018466 A1
(43) Pub. Date: Jan. 18, 2024

(54) METHOD FOR INDUCING SALMONELLA ENTERITIDIS INTO VBNC STATE BY SODIUM HYPOCHLORITE

(71) Applicants: Zhejiang Academy of Agricultural Sciences, Hangzhou (CN); China Jiliang University, Hangzhou (CN)

(72) Inventors: Wen Wang, Hangzhou (CN); Yingping Xiao, Hangzhou (CN); Hua Yang, Hangzhou (CN); Xingning Xiao, Hangzhou (CN); Wentao LV, Hangzhou (CN); Lingyan Ma, Hangzhou (CN); Kun Pang, Hangzhou (CN)

(21) Appl. No.: 18/352,719

(22) Filed: Jul. 14, 2023

(30) Foreign Application Priority Data
Jul. 18, 2022 (CN) 202210841210.8

(51) Int. Cl. C12N 1/36; C12N 1/20; C12N 1/02; C12Q 1/04

(52) U.S. Cl. CPC C12N 1/36; C12N 1/20; C12N 1/02; C12Q 1/04; C12R 2001/42

(57) ABSTRACT

The disclosure discloses a method for inducing Salmonella enteritidis into VBNC state by sodium hypochlorite. The disclosure relates to the field of biotechnology, including preparing culture medium, which is BHI culture medium, and the culture medium is cooled to room temperature, and sterilized by ultraviolet for 15 min. The strain to be tested is incubated in BHI at 37° C. for 24 h in a sterile environment, and the bacterial cells are collected by centrifugation, washed with 0.85% (w/v) sterile physiological saline for 3 times, and then resuspended in 0.85% (w/v) sterile physiological saline to obtain 10⁹ CFU/mL bacterial suspension. The bacteria cells are treated with sodium hypochlorite stress and the VBNC cells are counted.

### CROSS REFERENCE TO RELATED APPLICATION

This patent application claims the benefit and priority of Chinese Patent Application No. 202210841210.8, entitled “Method for inducing Salmonella enteritidis into VBNC state by sodium hypochlorite” filed on Jul. 18, 2022, the disclosure of which is incorporated by reference herein in its entirety as part of the present application.

### TECHNICAL FIELD

The disclosure relates to the field of biotechnology, specifically a method for inducing Salmonella enteritidis into VBNC state by sodium hypochlorite.

### BACKGROUND ART

Salmonella enteritidis infection can cause gastroenteritis type, typhoid type, septicemia type, cold type and cholera type food poisoning. The main symptoms are acute gastroenteritis, accompanied by fever, chills, nausea, headache, general fatigue, diarrhea, vomiting and other symptoms. Sodium hypochlorite is one of the most common disinfection technologies used to control microbial risk in food industry processing. It is often used for disinfection of drinking water, fruits and vegetables, and used as disinfectant for sterilization and disinfection of food manufacturing equipment and utensils for the prevention and control of foodborne pathogens. At present, most studies focus on the lethal mechanism of live bacteria after the treatment of disinfectant stress, ignoring the reaction mechanism of pathogenic bacteria in viable but non-culture (VBNC) state. After the bacteria enter the VBNC state, the cells become smaller through morphological changes to enhance their survival ability. At present, the reported factors that can induce bacteria into VBNC state mainly include low temperature, high temperature, oligonutrition, low pH, light, oxidative stress, hydrogen peroxide, etc. No patent literature has reported how to use sodium hypochlorite to induce and detect the VBNC state of Salmonella enteritidis.

### SUMMARY

The purpose of the disclosure is to provide a method for inducing Salmonella enteritidis into VBNC state by sodium hypochlorite to solve the problems raised in the above background art.

In order to achieve the purpose, the disclosure provides the following technical scheme: a method for inducing Salmonella enteritidis (S. Enteritidis) into VBNC state by sodium hypochlorite, including the following steps:

Step 1: selection of strains

selecting S. Enteritidis CVCC 1806, where the S. Enteritidis CVCC 1806 is obtained from the National Center for Veterinary Culture Collection, stored below −80° C. and passed for 1-5 times.

Step 2: preparing the medium, cooling the medium to room temperature, then carrying out an ultraviolet sterilization for 15 min, where the medium is BHI medium.

Step 3: preparation of bacterial suspension

incubating the strain to be tested in BHI medium at 37° C. for 24 h in a sterile environment, collecting bacterial cells by centrifugation, washing the bacterial cells with 0.85% (w/v) sterile physiological saline for 3 times, and then resuspending the bacterial cells in 0.85% (w/v) sterile physiological saline to obtain 10′ CFU/mL bacterial suspension;

where the centrifugation is conducted at ambient temperature of 4° C. with the centrifugation rate of 10000 rpm for 5 min.

Step 4: sodium hypochlorite stress treatment

diluting stock solution of NaClO disinfectant solution containing 56.8 mg/mL chlorine to 100 mg/L with sterilized sterile water for later use. Taking 0.5 ml of the obtained bacterial suspension (10⁹ CFU/mL) and the NaClO solution (100 mg/L) respectively into a sterile EP tube, and vortex-mixing products in the sterile EP tube. The stress concentration in the final system is 50 mg/L.

The NaClO disinfectant solution is available directly from the manufacturer or other commercial channels. The drug should be labeled with the generic name, batch number, concentration and expiry date thereof, and should be stored at the specified temperature and humidity. When it is diluted, it should be measured with the Palinstest chlorine analyzer (Gateshead, UK).

The uniformly mixed reagent is placed in a room temperature environment, and systems were obtained for processing every 0.5 h within a stress treatment for up to 3 h, and 100 u L 1.5% Na₂S₂O₃ is added to terminate the reaction.

Step 5: counts of VBNC state cells

counts of culturable cells: determining the number of culturable bacteria in the obtained system after stress treatments with 50 mg/L NaClO for different times in step 4 by plate counting method, continuously diluting 1 mL of disinfection sample with 0.85% (w/v) sterile physiological saline, and inoculating the obtained diluent in proper volume on the PCA plate, and then culturing at 37° C. for 24 h.

Counts of live cells: adjusting the concentration of bacterial suspension to 10⁷-10⁸ CFU/mL, and distinguishing live, dead and injured bacteria using commercial Live and Dead BacLight test kits (Molecular Probes, Carlsbad, CA, USA). Specifically, mixing SYTO9 dye and propidium iodide (PI) dye in equal volume, adding 3 μL mixed dye per 1 mL of disinfectant treated bacterial solution, adding 1 mL of bacterial solution heated at 90° C. for 15 min to 3 μL of PI dye and mixing them as a negative control, adding 1 mL of bacterial solution without disinfectant treatment to 3 μL of SYTO9 dye as a positive control, incubating the obtained mixture at room temperature and in dark for 15 min, filtering the incubated sample through a flow tube, detecting the obtained bacterial samples by using BD Biosciences FACS Verse flow cytometer (BD Biosciences, USA), and conducting FCM test using BD-AccuriC6 flow cytometer equipped with 488 nm and 640 nm laser.

To sum up, the beneficial effects of the technical scheme of the disclosure are:
1. According to the disclosure, the bacteria is induced into the VBNC state by sodium hypochlorite, avoiding the conventional dependence on low temperature, high temperature, oligonutrition, low pH, light, oxidative stress, hydrogen peroxide, etc. At the same time, the dye is adopted in counting method, which is more convenient and fast, and the observation effect is obvious.

### BRIEF DESCRIPTION OF THE DRAWINGS

In order to explain the examples herein or the technical solutions in the prior art more clearly, the following will briefly introduce the drawings needed in the embodiments or the description of the prior art. It is obvious that the drawings in the following description only describe some embodiments of the technical solutions. For those of ordinary skill in the art, other drawings can also be obtained from these drawings without creative work.

FIG. 1 shows the number of cells after NaClO solution treatment for different times herein;

FIGS. 2A-F show the flow cytometry after the solution treatment for different times herein.

### DETAILED DESCRIPTION OF THE EMBODIMENTS

All features disclosed in this specification, or all methods or steps in the process disclosed, can be combined in any way except for mutually exclusive features and/or steps.

Any feature disclosed in this specification (including any attached claims, abstract and drawings) can be replaced by other equivalent or alternative features with similar purpose unless otherwise stated. That is, unless otherwise stated, each feature is only one embodiment of a series of equivalent or similar features.

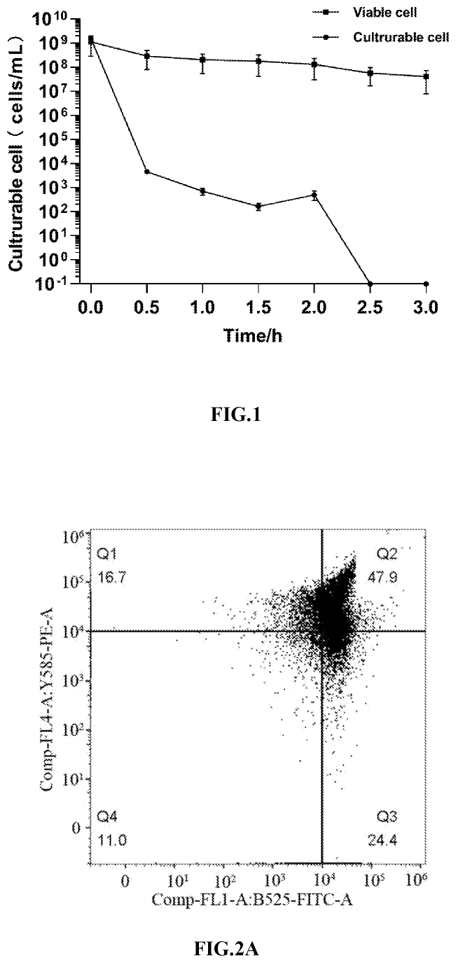

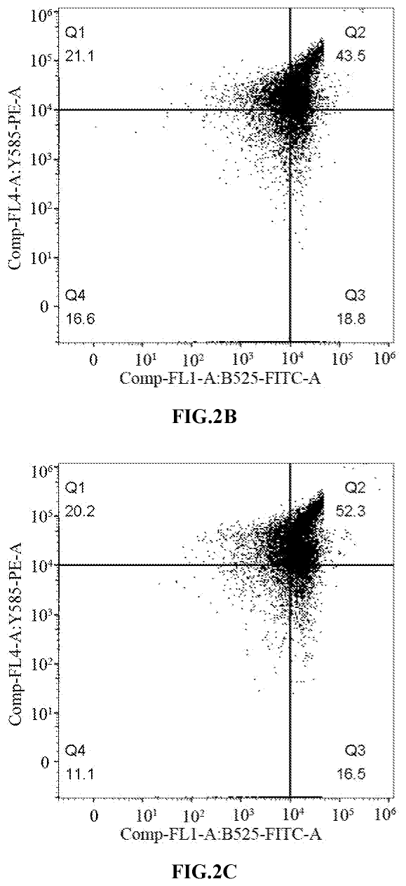

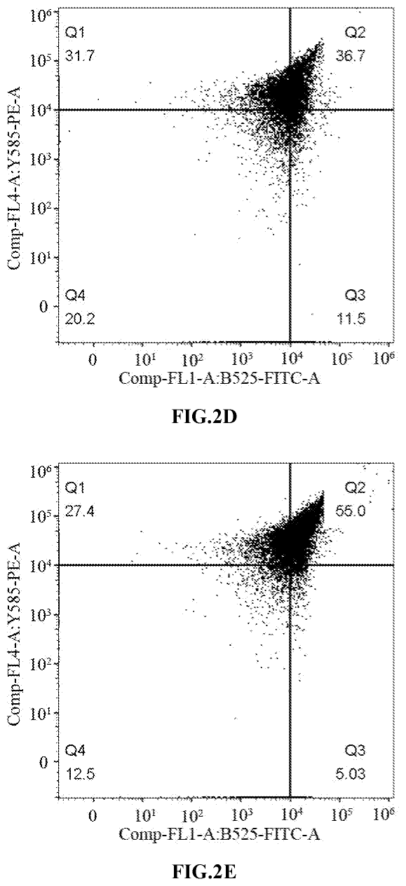

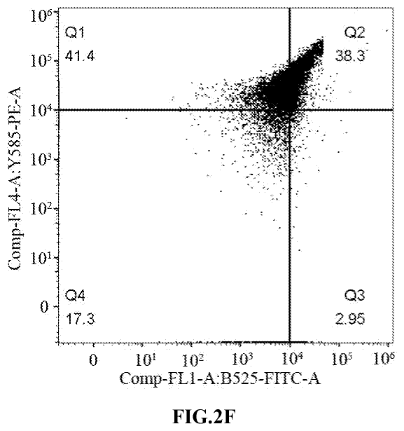

The technical solutions of the present disclosure is described in detail with reference to FIGS. 1-2. For the convenience of description, the following directions are specified as follows: the directions of the front, back, left, right, top, bottom are consistent with the directions of front, back, left, right, top, bottom of the view direction of FIG. 1. FIG. 1 is the front view of the device herein, and the directions shown in FIG. 1 are consistent with the directions of the front, left, right, top and bottom when facing the device herein.

Please refer to FIGS. 1-2, Example 1 is provided herein: a method for inducing Salmonella enteritidis into VBNC state by sodium hypochlorite, including the following steps:

step 1: selection of strains

S. Enteritidis CVCC 1806, obtained from China Microbiological Culture Collection Center, stored below −80° C. and passed for 1-5 times, was selected;

step 2: the medium, BHI medium was prepared, cooled to room temperature and subjected to an ultraviolet sterilization for 15 min;

step 3: preparation of bacterial suspension

the strain to be tested was incubated in BHI medium at 37° C. for 24 h in a sterile environment, and the bacterial cells were collected by centrifugation, washed with 0.85% (w/v) sterile physiological saline for 3 times, and then resuspended in 0.85% (w/v) sterile physiological saline to obtain 10′ CFU/mL bacterial suspension;

where the centrifugation was conducted at ambient temperature of 4° C. with the centrifugation rate of 10000 rpm for 5 min;

step 4: Sodium hypochlorite stress treatment

the NaClO stock solution of NaClO disinfectant solution containing 56.8 mg/mL chlorine was diluted to 100 mg/L with sterilized sterile water for later use, and 0.5 ml of bacterial suspension (10⁹ CFU/mL) and 100 mg/L NaClO solution were taken respectively into a sterile EP tube. The products in the sterile EP tube were vortex-mixed, and the stress concentration in the final system was 50 mg/L.

The NaClO disinfectant solution was available directly from the manufacturer or other commercial channels. The drug should be labeled with the generic name, batch number, concentration and expiry date of the thereof, and should be stored at the specified temperature and humidity. When it was diluted, it should be measured with the Palinstest chlorine analyzer (Gateshead, UK);

The mixed reagent was placed at room temperature, and systems were obtained for processing every 0.5 h within a stress treatment for up to 3 h, and 100 μL 1.5% Na₂S₂O₃ was added to terminate the reaction.

Step 5: counts of VBNC state cells

Counts of culturable cells: number of the culturable bacteria in the obtained system after stress treatment with 50 mg/L NaClO for different time in Step 4 was determined by plate counting method. 1 mL of disinfection sample was continuously diluted with 0.85% (w/v) sterile physiological saline, and the obtained diluent in proper volume was inoculated on the PCA plate, and then cultured at 37° C. for 24 h.

Counts of live cells: the concentration of bacterial suspension was adjusted to 10⁷-10⁹ CFU/mL, and live, dead and injured bacteria were distinguished using commercial Live and Dead BacLight test kits (Molecular Probes, Carlsbad, CA, USA). Specifically, SYTO9 dye and propidium iodide (PI) dye were mixed in equal volume, and 3 μL mixed dye was added to per 1 mL of disinfectant treated bacterial solution, 1 mL of bacterial solution heated at 90° C. for 15 min was added to 3 μL of PI dye and mixed as a negative control, 1 mL of bacterial solution without disinfectant treatment was added to 3 μL of SYTO9 dye and mixed as a positive control, and the obtained mixture was incubated at room temperature and in dark for 15 min, the incubated sample was filtered through a flow tube, the obtained bacterial samples was detected by using BD Biosciences FACS Verse flow cytometer (BD Biosciences, USA), and the FCM test was conducted by using BD-AccuriC6 flow cytometer equipped with 488 nm and 640 nm laser.

Further, in the Example, it can be seen from FIG. 1 that after 2.5 h of treatment with 50 mg/L NaClO solution, the number of culturable cells is 0 CFU/ml. Combined with flow cytometry analysis, after 2.5 h of treatment, the total content of live cells and bacteria in the sub-lethal injury state in the system is 60.03%. At this time, the difference between the number of live cells and the number of culturable cells reaches the maximum. The live and sub-lethal injury of Salmonella enteritidis in the system enter the VBNC state.

Further, in another Example, FIGS. 2A-F show the flow cytometry of treatment with 100 mg/L NaClO solution for 0.5 h, 1 h, 1.5 h, 2 h, 2.5 h and 3 h respectively;

where Q1: PI stained dead cells;

Q2: SYTO9 and PI stained injured cells;

Q3: SYTO9 stained living cells with intact cell membrane;

Q4: unstained cells.

The above is only the specific embodiments of the disclosure, but the protection scope herein is not limited to this. Any modification or replacement without creative work should be covered within the claimed scope of the disclosure. Therefore, the protection scope herein shall be subject to the protection scope defined in the claims.

What is claimed is:

1. A method for inducing Salmonella enteritidis (S. Enteritidis) into viable but non-culture (VBNC) state by sodium hypochlorite, which comprises the following steps:
step 1: selection of strains
selecting S. Enteritidis CVCC 1806, wherein the S. Enteritidis CVCC 1806 is obtained from the China Microbiological Culture Collection Center, stored below −80° C. and passed for 1-5 times;
step 2: preparing culture medium, cooling the medium to room temperature, then carrying out an ultraviolet sterilization for 15 min, where the medium is BHI medium;
step 3: preparation of bacterial suspension
washing bacterial cells with 0.85% (w/v) sterile saline for 3 times, collecting the bacterial cells by centrifugation, and then resuspending the bacterial cells in 0.85% (w/v) sterile saline to obtain 10¹ CFU/mL bacterial suspension;
step 4: sodium hypochlorite stress treatment
diluting NaClO stock solution of NaClO disinfectant solution containing 56.8 mg/mL chlorine to 100 mg/L with sterilized sterile water for later use, taking 0.5 ml of the obtained bacterial suspension (10⁹ CFU/mL) and the 100 mg/L NaClO solution respectively into a sterile EP tube, and vortex-mixing products in the sterile EP tube, and the stress concentration in the final system is 50 mg/L;
step 5: counts of VBNC state cells
counting culturable cells, determining number of the culturable bacteria in system after stress treatment with 50 mg/L NaClO for different times in step 4 by plate counting method;
counting live cells, adjusting the concentration of bacterial suspension to 107-108 CFU/mL, and distinguishing live, dead and injured bacteria using commercial live/dead staining kit (BacLight, L7012, Molecular Probes, Carlsbad, CA, United States).

2. The method for inducing Salmonella enteritidis into VBNC state by sodium hypochlorite according to claim 1, wherein in step 3, the centrifugation is conducted at ambient temperature of 4° C. with the centrifugation rate of 10000 rpm for 5 min.

3. The method for inducing Salmonella enteritidis into VBNC state by sodium hypochlorite according to claim 1, wherein in step 4, a uniformly mixed reagent is placed in a room temperature environment, and the processes of the stress treatment for 0.5 h, 1 h, 1.5 h, 2 h, 2.5 h and 3 h are recorded respectively, and 100 μL 1.5% Na₂S₂O₃ is added to terminate the reaction.

4. The method for inducing Salmonella enteritidis into VBNC state by sodium hypochlorite according to claim 1, wherein the counting of culturable cells in step 5 further comprises continuously diluting 1 mL of sterile sample with 0.85% (w/v) sterile physiological saline, and inoculating the obtained diluent in proper volume on the PCA plate, and then culturing at 37° C. for 24 hours.

5. The method for inducing Salmonella enteritidis into VBNC state by sodium hypochlorite according to claim 1, wherein, in step 5 further comprises the following steps:
S5.2.1 preparing dyes;
S5.2.2 preparing control reagent;
S5.2.3 detecting bacterial samples;
S5.2.4 conducting a flow cytometry (FCM) test.

6. The method for inducing Salmonella enteritidis into VBNC state by sodium hypochlorite according to claim 5, wherein a step of preparing dyes comprises mixing SYTO9 dye and propidium iodide (PI) dye in 1:1 volume.

7. The method for inducing Salmonella enteritidis into VBNC state by sodium hypochlorite according to claim 5, wherein the control reagent comprises negative control reagent and positive control reagent, the negative reagent is prepared by adding 3 μL mixed dye per 1 mL of disinfectant treated bacterial solution, adding 1 mL of bacterial liquid heated at 90° C. for 15 min to 3 μL of PI dye, and mixing an obtained mixture; and the positive reagent is prepared by adding 1 mL of bacterial liquid without disinfectant treatment to 3 μL of SYTO9 dye and mixing an obtained mixture.

8. The method for inducing Salmonella enteritidis into VBNC state by sodium hypochlorite according to claim 5, wherein a step of detecting bacterial sample comprises incubating mixture at room temperature and in dark for 15 min, filtering an incubated sample through a flow tube, and detecting the detect bacterial samples by using BD Biosciences FACS Verse flow cytometry (BD Biosciences, USA).

9. The method for inducing Salmonella enteritidis into VBNC state by sodium hypochlorite according to claim 5, wherein the FCM test is performed by using BD-AccuriC6 flow cytometer equipped with 488 nm and 640 nm laser.
